(19) United States
(12) Patent Application Publication
(10) Pub. No.: US 2013/0190671 A1
(43) Pub. Date: Jul. 25, 2013

(54) Device for correcting an underlapping or overlapping toe or finger

(71) Applicant: John Pastore, Warwick, RI (US)

(72) Inventor: John Pastore, Warwick, RI (US)

(21) Appl. No.: 13/374,945

(22) Filed: Jan. 24, 2012

(51) Int. Cl. A61F 5/10

(52) U.S. Cl. 602/22; 602/30

(57) ABSTRACT

In accordance with the invention, briefly stated, a toe brace or support comprises a first sleeve and a second sleeve of elastic material joined at a peripheral edge. The first sleeve fits over the overlapping toe and the second sleeve is stretched under an adjacent toe and positioned onto a third toe for an overlapping toe condition. For an underlapping condition, the first sleeve fits over the underlapping toe and the second sleeve is stretched over an adjacent toe and then positioned onto the third toe. The device utilizes the force provided by having the second sleeve stretched to skip over an adjacent toe onto a third toe. The invention could equally be applied to fingers if desired.

### FIELD OF THE INVENTION

The present invention relates to the treatment of an underlapping or overlapping toe or finger, in particular to a toe or finger correction device which is conveniently worn by the person with the deformity to correct the deformity.

### BACKGROUND OF THE INVENTION

A common toe deformity refers to a condition that a big toe leans towards a second toe, and a first toe joint of the big toe is deformed and protruded, and such condition is generally related to a congenital genetic factor or an acquired abnormal force applied to a foot. In these circumstances the big toe can either overlap or underlap an adjacent toe. Particularly for a person who wears high heels, pointed shoes, or too-narrow shoes for long time, such that the wearer's body weight concentrates at the front end of the foot during walking and destroys the function of the original three foot arcs, and the big toe and other toes may be deformed gradually due to the wearer's body weight. When this happens, the big toe and other toes may be rubbed and compressed to affect the stretch and movement of the toes. Compounding the problem, when shoes are worn the toes are further restricted causing calluses and corns over a period of time.

However, general toe deformities can be corrected by products known in the art for alignment of the toes utilizing complicated apparatus that may be inconvenient to wear on a daily basis.

The present invention provides a device for maintaining the alignment of the big toe, or any toe that is underlapping or overlapping, which can be worn on a daily basis while wearing shoes and socks. The invention may equally apply to similar deformities of the fingers.

### SUMMARY OF THE INVENTION

In accordance with the invention, briefly stated, a toe brace or support comprises a first sleeve and a second sleeve of elastic material joined at a peripheral edge. The first sleeve fits over the overlapping toe and the second sleeve is stretched under an adjacent toe and positioned onto a third toe for an overlapping toe condition. For an underlapping condition, the first sleeve fits over the underlapping toe and the second sleeve is stretched over an adjacent toe and then positioned onto the third toe. The device utilizes the force provided by having the second sleeve stretched to skip over an adjacent toe onto a third toe. The invention could equally be applied to fingers if desired.

The adjacent toe provides a force acting downward between the first and second sleeve (for overlapping toes) and a force acting upward between the first and second sleeve (for underlapping toes). The tension produced between the first and second sleeve by the adjacent toe for both overlapping and underlapping conditions produces an action force to push away the deformed toe into straight alighnment.

The device can be left in place as long as desired, does not unduly restrict activities of the patient, and for persons suffering from a deformed toe, allows the patient to wear normal shoes and socks. The support is low cost, re-usable, and is available in different sizes and shapes. Other objects and features will be in part apparent and in part pointed out hereinafter.

The invention is best understood from the following detailed description when read in connection with the accompanying drawings, which illustrate various embodiments of the present invention:

### BRIEF DESCRIPTION OF THE DRAWINGS

FIG. 1 is a front view of the device of the present invention.

FIG. 2 is a top view of the device of FIG. 1.

FIG. 3 is an illustration of a human foot illustrating an overlapping toe condition.

FIG. 4 is an illustration of a human foot illustrating an underlaping toe condition.

FIG. 5 is an illustration of the device of the present invention implemented to correct an overlapping toe condition.

FIG. 6 is an illustration of the present invention implemented to correct an underlapping toe condition.

FIG. 7 illustrates a front view of FIG. 5.

FIG. 8 illustrates a front view of FIG. 6.

FIG. 9 illustrates the device of FIG. 1 in a stretched condition.

### DETAILED DESCRIPTION OF THE INVENTION

The following detailed description illustrates the invention by way of example and not by way of limitation. This description will clearly enable one skilled in the art to make and use the invention, and describes embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

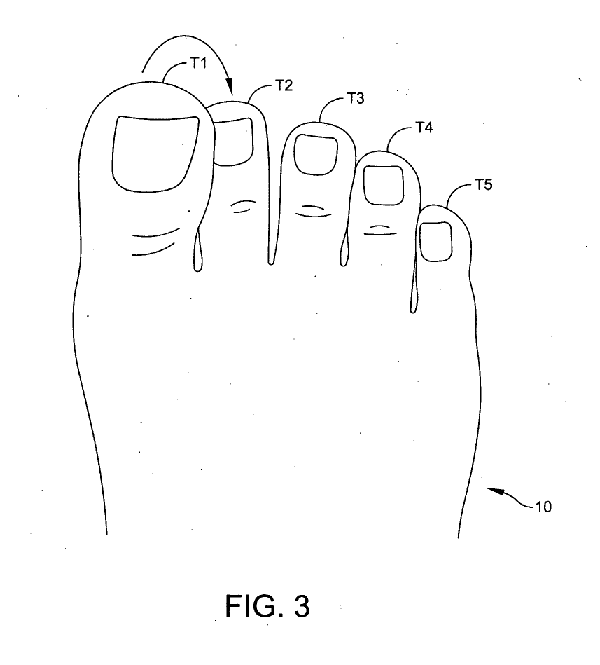

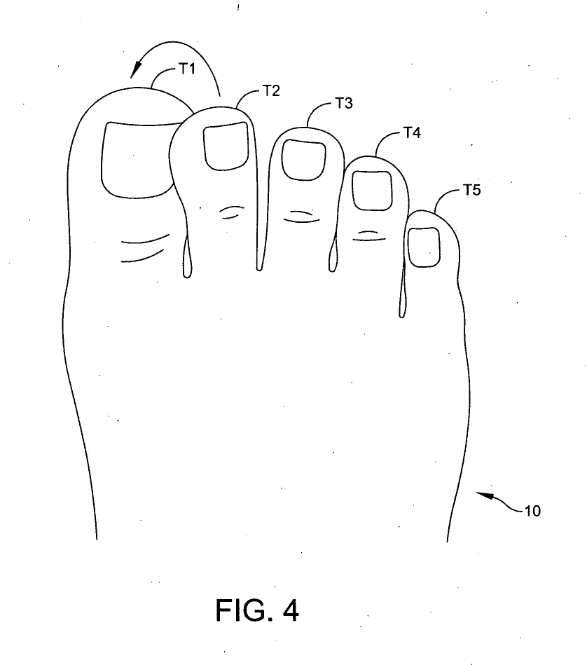

Referring to the drawings, the present invention is directed to the correction of a deformed digit, whether the digit be a person's toe, or a finger of their hand. FIGS. 3 and 4 illustrate deformities to the toes of a foot 10. FIG. 3 and FIG. 4 illustrates the condition of overlapping and underlapping toe toe T1, rather than extending in a side-by-side, generally parallel relationship with toe T2, overlaps (FIG. 3) or underlaps (FIG. 4) an adjacent toe of the foot. It is understood that the underlapping or overlapping toe could be any toe T1-T5.

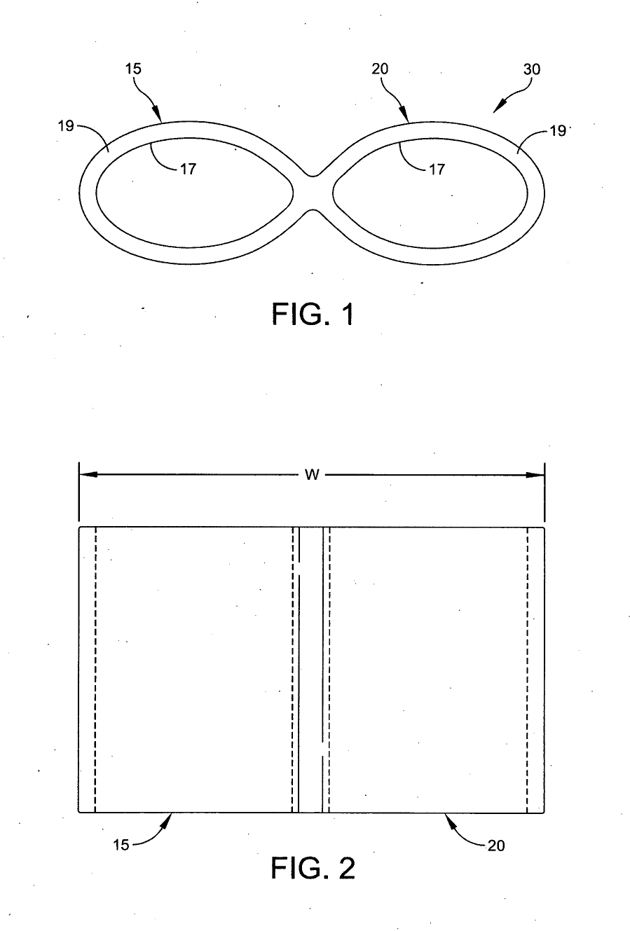

While not shown in the drawings, similar deformities occur with the fingers. It will be understood that that while the deformity shown is a common deformity, other conditions to the toes and fingers are also correctable using the supports of the present invention. In the embodiments described hereinbelow, it will be understood by those skilled in the art, that the device of the present invention is readily adapted for wearing by someone suffering from a similar type finger deformity. Significant advantages to the invention are its low cost, providing the patient with a comfortable support for a painful condition; and, for patient's with toe deformities, allowing the patient to wear ordinary shoes and socks and engage in a wide range of activities in which they might not otherwise be able to participate. In accordance with the invention, as illustrated in FIG. 1, a support 30 is provided which is worn on the patient's foot to correct the deformity. The support 30 comprises a first sleeve 15 and a second sleeve 20 joined along a longitudinal length, preferably at a peripheral edge. Sleeves 15 and 20 are made of an elastic material and may further include a layer of padding on the underside of sleeves 15, 20, to prevent irritation. In the preferred embodiment, sleeves 15, 20 include the elastic material 17 and an outer layer of padding 19.

In addition to its elasticity, the material is also preferably wrinkle free. This prevents the toes from being irritated, when the support 30 is worn, and particularly prevents sores. The material from which the support 30 is made may be washable so the support can be cleaned after wearing and reused. Alternately, the support 30 is disposed of after being worn.

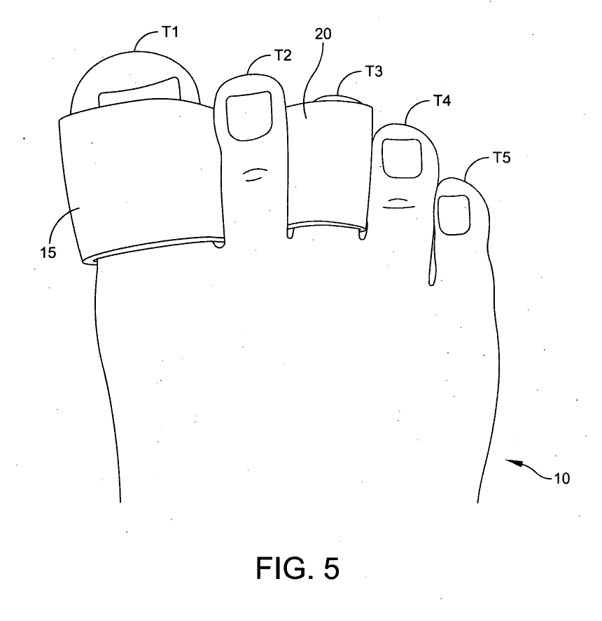

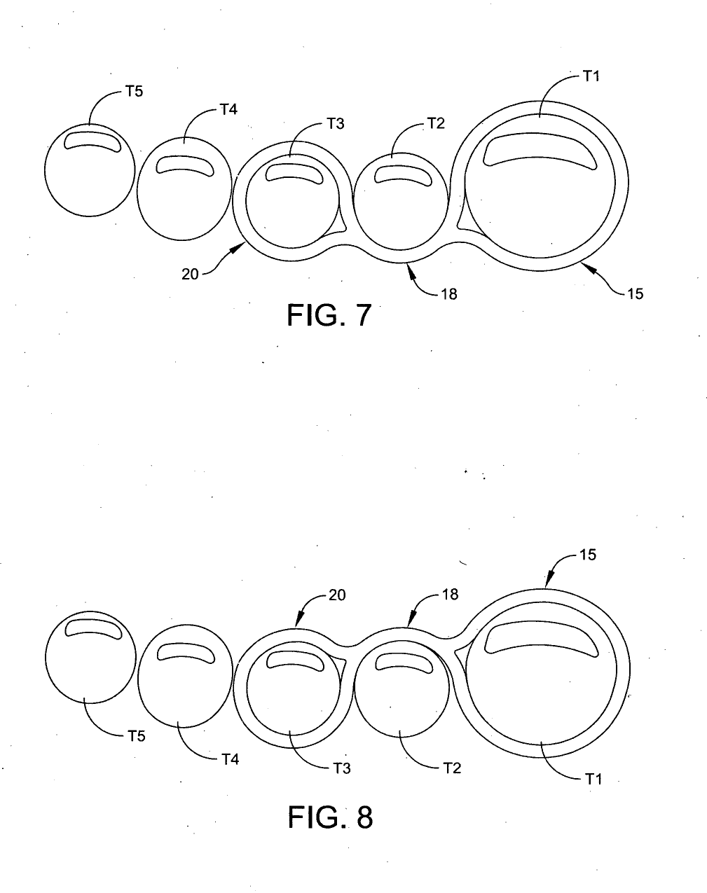

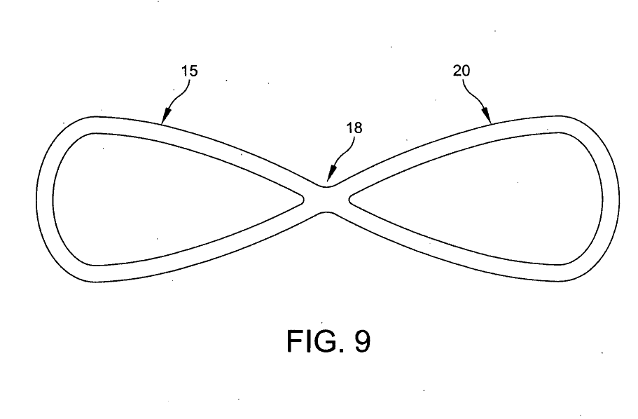

FIG. 2 illustrates a top view of support 30 which in the preferred embodiment measures approximately 2.5 inches in width w. This width can be varied according to application and distance between a users toes. In the preferred embodiment, the width was chosen to accommodate an adult user with either overlapping or underlapping big toe T1. As illustrated in FIGS. 5 and 7, for an overlapping toe condition as illustrated in FIG. 3, first sleeve 15 is placed over big toe T1, and second sleeve 20 is stretched under toe T2 and placed onto toe T3. Toe T2 may include a separate detached sleeve thereon if desired.

The elasticity of first and second sleeves 15 and 20 combined with the downward force produced by toe T2 onto the center 18 (see FIG. 3) of a stretched support 30 produces a tension to produce an action force that pushes away the deformed toe T1 into straight alignment.

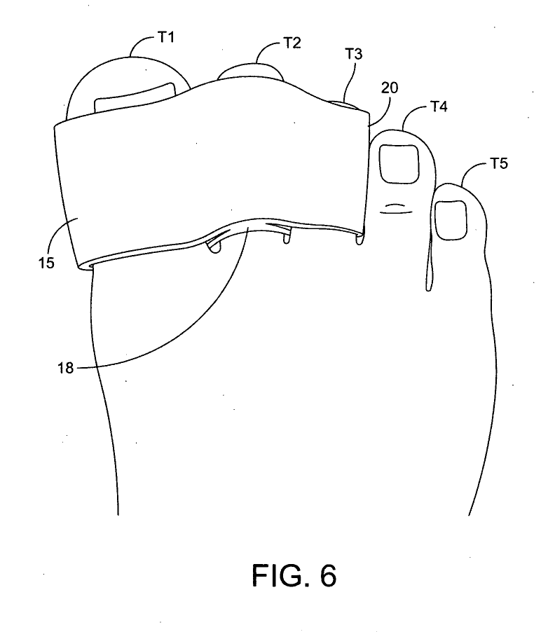

As illustrated in FIGS. 6 and 8, for an underlapping toe condition as illustrated in FIG. 4, first sleeve 15 is placed over big toe T1, and second sleeve 20 is stretched over toe T2 then placed onto toe T3.

The elasticity of first and second sleeves 15 and 20 combined with the upward force produced by toe T2 onto the center 18 (see FIG. 3) of a stretched support 30 produces a tension to produce an action force that pushes away the deformed toe T1 into straight alignment.

The device utilizes the force provided by having the second sleeve stretched to skip over an adjacent toe onto a third toe. The invention could equally be applied to fingers if desired.

The stretched support 30 suppresses the distortion of the toe so when the support is in place, the deformed toe is held in place in a more normal side-by-side toe position rather than overlying an adjacent toe. The material is elastic, and relies on an adjacent toes force to keep the affected toe from assuming its deformed position.

More force may be required to keep the deformed toe in a side-by-side position extending generally parallel with the other toes.

The force exerted on the deformed toe to straighten it can be controlled by the type of material from which elastic material 17 is made. Depending upon the elasticity of the material 17, more or less force is exerted on the deformed toe. The less elastic the material, the more force is placed upon the deformed toe to maintain it straight.

Support 30 is useful to correct deformation of any toe. As previously described, first and second sleeves, 15 and 20, could be placed on any combination of toes, T1-T5, provided center 18 of support 30 is place under (overlapping toe) or over (underlapping toe) the toe adjacent to the deformed toe.

While not shown, those skilled in the art will understand that other toe supports within the scope of this invention are possible. In view of the foregoing, it will be seen that the several objects of the invention are achieved and other advantageous results are obtained.

As various changes could be made in the above constructions without departing from the scope of the invention, it is intended that all matter contained in the above description or shown in the accompanying drawings shall be interpreted as illustrative and not in a limiting sense.

I claim:

1. A support worn by a person on their hand or foot to correct a deformity to a digit (finger or toe) of the hand or foot, comprising: a first sleeve of a support material sized to fit over a deformed digit; a second sleeve of the material sized to fit over another digit, said another digit positioned two digits over from said deformed digit, said second sleeve attached to said first sleeve, said second sleeve stretched to fit over said another digit, forming a stretched second sleeve, said stretched second sleeve producing a force on said attached first sleeve forcing said deformed digit in a preferred position in which it extends generally side-by-side with the other digits, said first and said stretched second sleeve attached at their periphery, said stretched first and second sleeves forming a band portion therebetween at said attached periphery.

2. (canceled)

3. The support of claim 1, wherein said band portion extends under a middle digit, said middle digit one digit over from said deformed digit, said force on said attached first sleeve pushing said deformed digit downward to correct an overlapping toe condition.

4. The support of claim 1, wherein said band portion extends over a middle digit, said middle digit one digit over from said deformed digit, said force on said attached first sleeve pushing said deformed digit upward to correct an underlapping toe condition.

5. (canceled)

6. The support of claim 1, in which the first and second sleeves are of the same diameter.

7. The support of claim 1, in which one of the sleeves is larger in diameter than the other.

8. The support of claim 1, in which the first and second sleeves are of the same length.

9. (canceled)

10. The support of claim 1, in which the outside of each sleeve is lined with a padding material to prevent irritation of the person's skin when wearing the support.

11. The support of claim 1, wherein the first and second sleeves include an elastic material to facilitate stretching.

12. (canceled)

13. (canceled)

14. A method of treating a deformed toe on a person's foot, comprising:
fitting a first sleeve over the deformed toe;
Stretching an attached second sleeve to skip over an adjacent toe and fit over a third toe.

15. The method as in claim 14, further including the step of stretching said second sleeve under said adjacent toe to correct an overlapping toe condition.

16. The method as in claim 14, further including the step of stretching said second sleeve over said adjacent toe to correct an underlapping toe condition.

17. A method of treating a deformed finger on a person's hand, comprising: fitting a first sleeve over the deformed finger; stretching an attached second sleeve to skip over an adjacent finger and fit over a third finger.

18. The method as in claim 17, further including the step of stretching said second sleeve under said adjacent finger to correct an overlapping finger condition.

19. The method as in claim 17, further including the step of stretching said second sleeve over said adjacent finger to correct an underlapping toe condition.

20. A support worn by a person on their hand or foot to correct a deformity to a digit (finger or toe) of the hand or foot, comprising:
a first sleeve of a support material sized to fit over a deformed digit;
a second sleeve of said support material attached to said first sleeve, said first and second sleeves stretchable outward from said attachment, said second sleeve stretched to fit over another digit, forming a stretched second sleeve, said stretched second sleeve producing a force on said attached first sleeve forcing said deformed digit in a preferred position in which it extends generally side-by-side with the other digits.
